(19) United States
(12) Patent Application Publication
(10) Pub. No.: US 2026/0096728 A1
(43) Pub. Date: Apr. 9, 2026

(54) STRESS ESTIMATION DURING OPHTHALMIC TREATMENTS

(71) Applicant: Alcon Inc., Fribourg (CH)

(72) Inventors: Reto GRÜEBLER, Greifensee (CH); Niccolo MASCHIO, Winterthur (CH)

(21) Appl. No.: 19/351,126

(22) Filed: Oct. 6, 2025

Related U.S. Application Data
(60) Provisional application No. 63/705,110, filed on Oct. 9, 2024.

(51) Int. Cl. A61B 3/13; A61B 3/00

(52) U.S. Cl. CPC A61B 3/13; A61B 3/0025; A61B 3/0041

(57) ABSTRACT

An ophthalmic surgical system includes an ophthalmic microscope including a first camera and a second camera. A controller coupled to the first camera and the second camera receives a current pair of images including a current representations of a retina of a patient; obtains a current three-dimensional representation of the retina from the current pair of images; determines deformation of the retina by evaluating the current three-dimensional representation relative to a reference representation of the retina; determines stress on the retina according to the deformation; and outputs guidance according to the stress. Stress may be determined using FEM and may use velocity and acceleration of the deformation. Guidance may include a graphical representation of the stress or a vector along which force should be exerted. Deformation may be detected using a mesh projected onto the retina.

### CROSS-REFERENCE TO RELATED APPLICATIONS

This application claims the benefit of U.S. Provisional Patent Application No. 63/705,110, filed October 9, 2024, which is incorporated by reference herein in its entirety, and is hereby expressly made a part of this specification.

### INTRODUCTION

The present disclosure relates generally to estimating stress during ophthalmic surgery, such as stress on the retina during membrane peeling, vitrectomy, retinal detachment repair (e.g., for proliferative vitreoretinopathy (PVR) or diabetic membranes).

The human eye receives light through a clear outer portion called the cornea and focuses the resulting image by way of an ocular crystalline lens onto the retina. The volume of the eye between the lens and the retina is occupied by a clear gel known as the vitreous. A thin film known as the internal limiting membrane (ILM) separates the retinal from the vitreous. Other pathological membranes may also form over the retina, such as an epiretinal membrane (ERM), proliferative vitreoretinopathy (PVR), diabetic membrane, or free floating retina or retinal bleb in the case of a detached retina. Some conditions require removal of the ILM and an ERM may also need to be removed to restore visual acuity. Still other conditions require the vitreous to be removed. Many of these procedures may place stress on the retina. Accordingly, it is important to ensure that such procedures do not place undue stress on the retina and cause injury.

### SUMMARY

In certain embodiments, a system includes an ophthalmic microscope including a first camera and a second camera. The system further includes a controller coupled to the first camera and the second camera. The controller is configured to: receive a current pair of images from the first camera and the second camera, the current pair of images including a current representations of a retina of a patient; obtain a current three-dimensional representation of the retina from the current pair of images; determine deformation of the retina by evaluating the current three-dimensional representation relative to a reference representation of the retina; determine stress on the retina according to the deformation; and output guidance according to the stress.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example operating environment for providing ophthalmic treatments, in accordance with certain embodiments.

FIG. 2 illustrates components of an ophthalmic microscope, in accordance with certain embodiments.

FIG. 3A to 3D illustrates using a mesh to determine stress on the retina, in accordance with certain embodiments.

FIG. 4A is a process flow diagram of a method for determining stress on the retina during an ophthalmic treatment, in accordance with certain embodiments.

FIG. 4B is a process flow diagram of an alternative method for determining stress on the retina during an ophthalmic treatment, in accordance with certain embodiments.

FIG. 5 illustrates an example computing device that implements, at least partly, one or more functionalities for determining stress on the retina during an ophthalmic treatment and facilitating visualization during ophthalmic surgery, in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

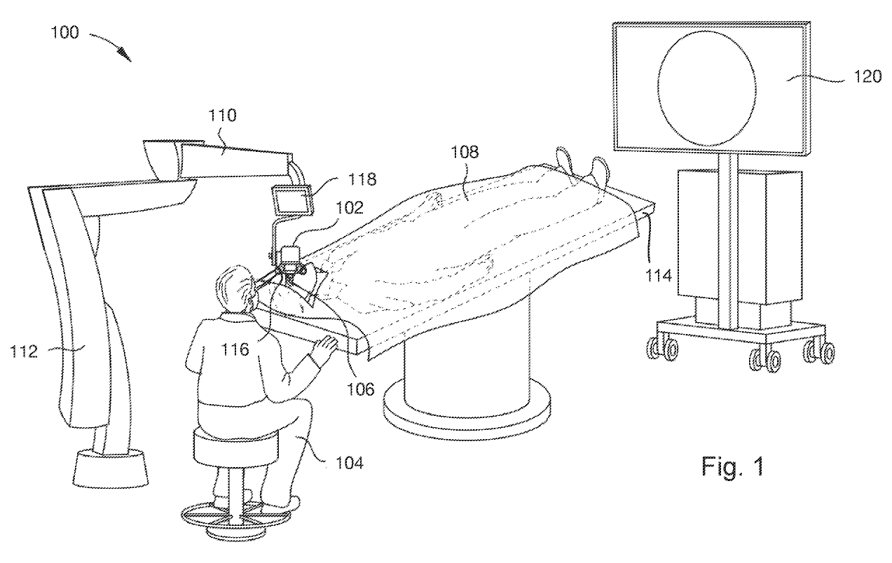

FIG. 1 illustrates an example operating environment including an ophthalmic surgical system 100 with which ophthalmic treatments may be performed. The ophthalmic surgical system 100 includes an ophthalmic microscope 102, used by a surgeon 104 to visualize structures on and in an eye 106 of a medical patient 108 in the field of view of the ophthalmic microscope 102. The ophthalmic microscope 102 is supported on, in this illustration, an adjustable overhead arm 110 of a microscope support pedestal 112. The patient 108 may be supported on an operating table 114. The ophthalmic microscope 102 is movable with the overhead arm 110 in three dimensions so that the surgeon 104 can position the ophthalmic microscope 102 as desired with respect to the eye 106 of the patient 108.

In certain embodiments, the ophthalmic microscope 102 comprises a high resolution, high contrast stereo viewing surgical microscope. The ophthalmic microscope 102 will often include a monocular eyepiece 116 or binocular eyepieces 116, through which the surgeon 104 will have an optically magnified view of the relevant eye structures that the surgeon 104 will need to see to accomplish a given surgery or diagnose an eye condition of the patient 108.

The ophthalmic microscope 102 includes a digital camera and a broadband light source for capturing color (red, green, and blue) images and/or infrared images. The ophthalmic microscope 102 may, in certain embodiments, further include a multi-spectral imaging (MSI) device, and/or other type of imaging device. Digital images captured using the camera may be displayed on a display device within the ophthalmic microscope 102.

The ophthalmic microscope 102 may include two display devices that are viewable through binocular eyepieces 116 and that display images of the patient’s eye 106 captured from different viewpoints by two cameras to provide stereoscopic viewing. For example, the ophthalmic microscope 102 may be implemented as the NGENUITY 3D VISUALIZATION SYSTEM provided by Alcon Inc. of Fort Worth Texas.

Images from the ophthalmic microscope 102 may be additionally or alternatively displayed on one or more display devices. For example, the one or more display devices may include a display device 118 fastened to the supporting arm 110 above the ophthalmic microscope 102.

In order to relieve the surgeon 104 from the need to constantly look into the eyepieces 116 to obtain a stereoscopic view, the one or more display devices may also include a display device 120 that can be implemented as a three-dimensional display device. The display device 120 may therefore provide a stereoscopic view of images captured using the ophthalmic microscope 102. The display device 120 may be embodied as any type of three-dimensional display device known in the art, including those that do or do not use special filtering glasses. For some types of three-dimensional display devices, the perception of three dimensions requires that the distance of the viewer from the display device 120 be within a threshold distance from the display device. The display device 120 may be mounted to a cart, a manually adjustable or robotic arm, or other manually or automatically adjustable support.

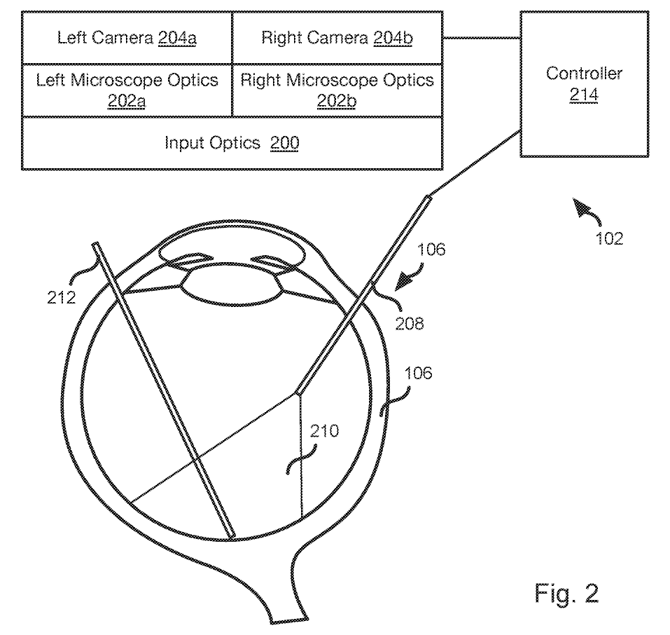

FIG. 2 is schematic diagram of the ophthalmic microscope 102 of FIG. 1. The ophthalmic microscope 102 may include input optics 200, left and right microscope optics 202a, 202b, and left and right cameras 204a, 204b. As used herein, “left” and “right” are used to refer to first and second instances of a component facilitating visualization by the left and right eyes of the surgeon 104. The use of “left” and “right” shall be understood as exemplary only and it shall be understood that these can be readily interchanged without change in functionality.

The input optics 200 receive light reflected from the eye 106 of the patient. The input optics 200 may be a set of lenses with a common optical axis or two sets of lenses with offset and/or non-parallel optical axes, e.g., right and left sets of lenses.

Light reflected from the eye 106 is magnified by left and right microscope optics 202a, 202b, respectively. The magnification of the left and right microscope optics 202a, 202b may be adjustable. Likewise, the depth of focus of the left and right microscope optics 202a, 202b may be adjustable. Light reflected from the eye is emitted by the left and right microscope optics 202a, 202b onto the left and right cameras 204a, 204b. The left and right cameras 204a, 204b output images that may be displayed on any of the display devices 118, 120, an internal display of the ophthalmic microscope 102, or other display device. The images output by the left and right cameras 204a, 204b may further be processed according to the methods described to detect retinal deformation.

An endoillumination probe 208 may be inserted into the eye 106, such as through the sclera or choroid. The endoillumination probe 208 directs a beam of light 210 onto the retina to illuminate the area of the retina being treated. An instrument 212, such as a scraper, vitrectomy tool, or other type of instrument may likewise be inserted into the eye 106 in order to treat the retina.

A controller 214 may be coupled to the cameras 204a, 204b to receive images from the camera. The controller 214 may further control the supply of light by the endoillumination probe 208. For example, the controller 214 may perform detection of deformation according to the methods discussed below.

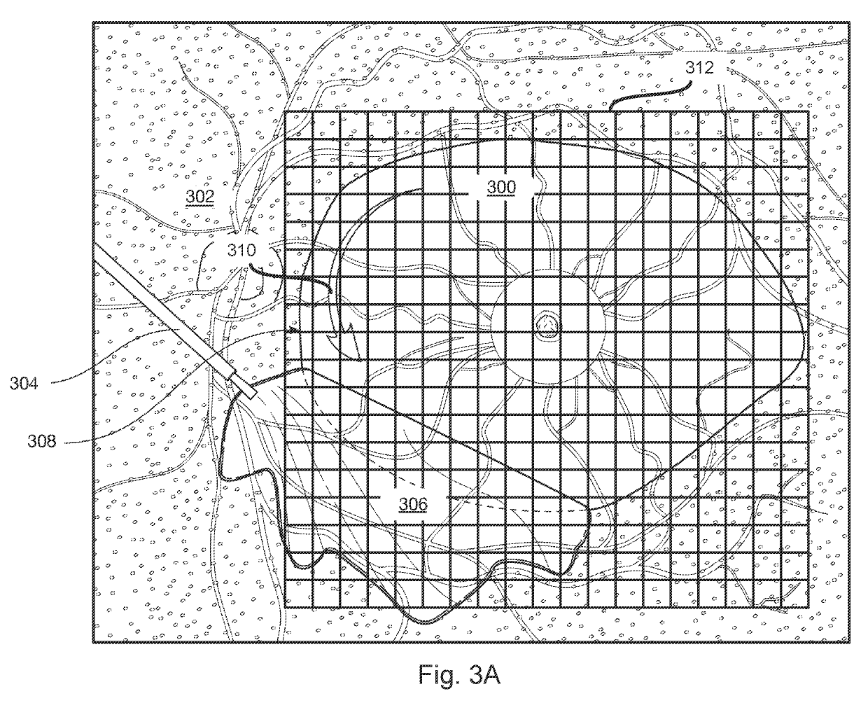

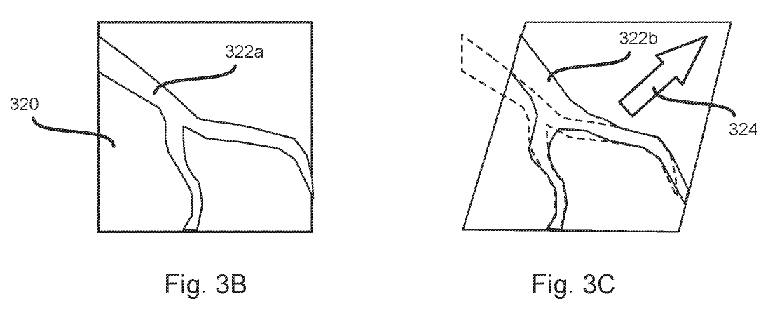

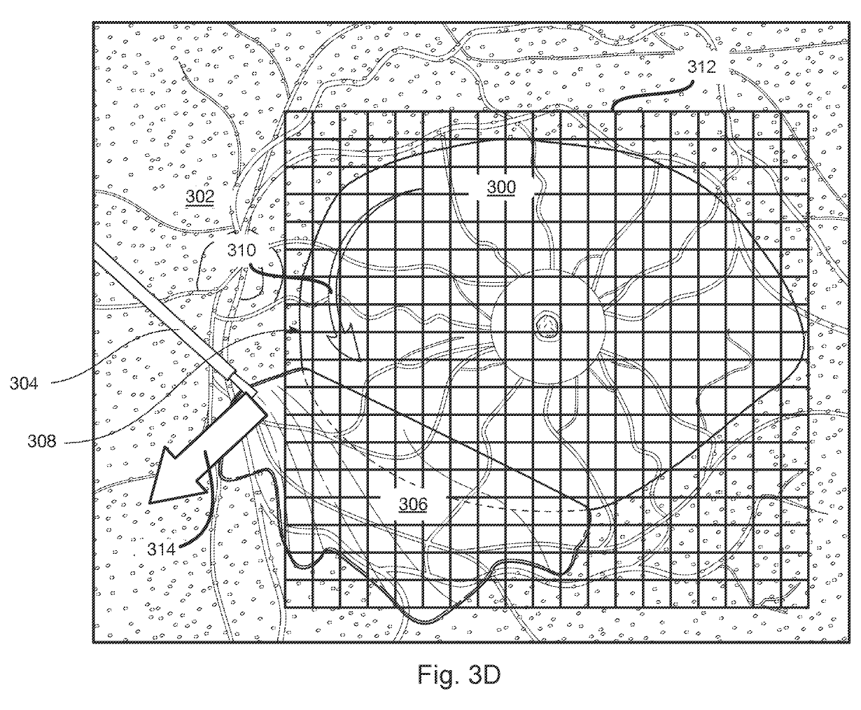

FIGS. 3A to 3D illustrate a first approach for detecting deformation of the retina, which may be used to determine stress on the retina. FIG. 3A illustrates an image that may be captured using the left camera 204a and the right camera 204b. In operation, images from the left camera 204a and the right camera 204b may be used to obtain three-dimensional information.

The image may include representations of the retina 300 and a membrane 302 formed on the retina 300, such as an ILM or an ELM. The image may show a portion of an instrument 304 that is used by the surgeon 104 to grasp a peeled portion 306 of the membrane 302 and remove a portion thereof over at least a region 308 of the retina 300, such as using the illustrated circular motion 310. An image of a vitrectomy tool or other type of instrument may be captured in a like manner.

There are various opportunities to induce stress on the retina 300 during a membrane peeling procedure. The instrument 304 may include forceps or a scraper that may press into the retina 300 in order to raise a flap that is subsequently grasped in order to perform peeling. During peeling, the peeled portion 306 may pull outwardly (e.g., in the normal direction) and/or laterally (e.g., in a tangent direction) on the retina 300.

Other procedures may also induce stress on the retina. During a vitrectomy, the vitreous is removed from the eye 106 using suction. Accordingly, both outward and lateral forces may be exerted on the retina 300. A scleral indentation includes deforming the globe of the eye from outside the eye, which may likewise induce stress on the retina. The above-listed examples are exemplary only. Retinal reattachment procedures may also benefit from estimating stress on the retina 300. Other ophthalmic procedures involving the retina 300 or other structures of the eye may induce stress on the retina 300. Accordingly, any such ophthalmic procedure may advantageously be performed along with the approach described herein.

In some embodiments, a mesh 312 may be defined virtually with respect to the retina, such as with respect to an image of the retina or with respect to geometry of the retina itself. The mesh 312 is shown with rows and columns of generally perpendicular lines (e.g., with some distortion due to sphericity of the retina 300). However, other approaches for creating a detectable pattern on the retina 300 may be used, such as an ordered or random array of points, a polar grid (concentric circles and radially extending lines), or other pattern.

FIGS. 3B and 3C illustrate an approach for detecting deformation of the retina using an image and a virtual mesh. The image may be an image from one of the left camera 204a and the right camera 204b, a set of images from the left camera 204a and the right camera 204b, or a three-dimensional image obtained from images from the left camera 204a and the right camera 204b.

Referring to FIG. 3B, representation 322a of features of the retina may be detected in a reference image, e.g., a preoperative image or intraoperative image captured prior to a more recently received image (“the current image”). Features may be blood vessels, points on the optic disc, variation in color, randomly selected groupings of pixels, or any other visually distinguishable feature. The representations 322a may be selected randomly or with reference to the mesh 312, e.g., one or more representations 322a per region 320 defined by the mesh 312, a region 320 being a rectangular defined between by the lines of the mesh 312.

Referring to FIG. 3C, representations 322b of the same features may be found in the current image. Deformation of the retina may result in distortion of features and movement of the features relative to one another and such deformation may be detected by comparing the reference and current images. The deformation of representations 322a in the first image with respect to the corresponding representations 322b in the second image may be used to determine strain on the retina. For example, for each region 320, a strain vector 324 for the region 320 may be calculated based on the distortion of one or more representations 322b within the region 320. Since the reference and current images may be three dimensional representations of the retina, the deformation may likewise be determined in three dimensions.

A field of strain vectors 324 may be generated for some or all of the regions 320 of the mesh 312. The field of strain vectors 324 may then be processed to determine the stress on the retina. For example, the strain vectors 324 along with assumed material properties of the retina (e.g., an experimentally determined modulus of elasticity) may be processed according to a finite element modeling (FEM) algorithm that provides a solution for stress (e.g., a corresponding field of stress vectors) in the retina. The FEM algorithm used may be an explicit algorithm that is guaranteed to converge.

Referring to FIG. 3D, the stress on the retina may be further processed to determine a pulling vector 314 (e.g., when peeling a membrane) that will reduce the stress on the retina to acceptable levels.

The approach of FIGS. 3A to 3D may be repeated throughout an ophthalmic treatment. For example, the current image may become the reference image for a subsequent iteration and a subsequently captured image may be used as the current image in the subsequent iteration.

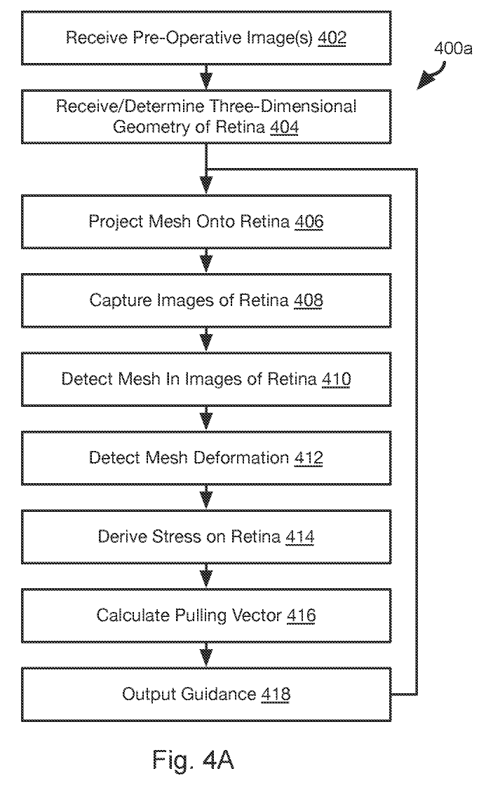

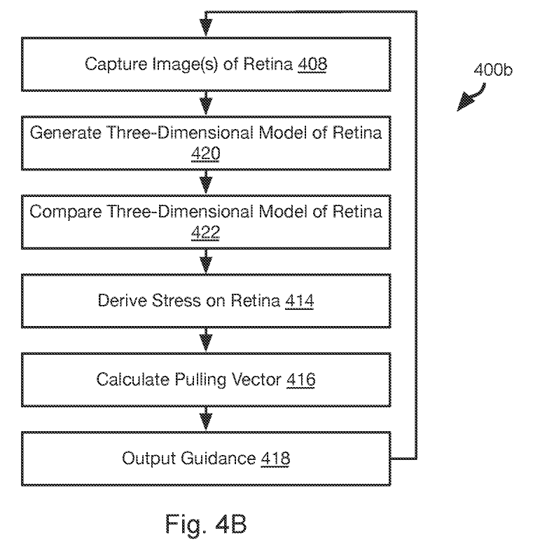

FIG. 4 illustrates a method 400a that may be performed in order to estimate stress on the retina 300 using the mesh 312 during an ophthalmic treatment. The method may be performed by the controller 214 and may be performed as part of or as an alternative to the approach described above with respect to FIGS. 3A to 3D. The method 400a may optionally include outputting guidance to the surgeon 104 based on the estimated stress. The method 400a may be performed using the ophthalmic microscope 102 with operations of the method 400a being performed by or invoked by the controller 214 or some other computing device.

The method 400a may include receiving, at 402, one or more pre-operative images of the retina. For example, the pre-operative images may be images captured using the left camera 204a and right camera 204b of the ophthalmic microscope 102 used during the ophthalmic treatment (e.g., prior to any deformation of the retina) or in a clinic using a different ophthalmic microscope.

The method 400a may include receiving, at 404, pre-operative three-dimensional geometry of the retina 300. Alternatively, 404 may include determining three-dimensional geometry of the retina 300 from the pre-operative images received at 402. The three-dimensional geometry of the retina 300 may include a volumetric image of the retina 300, a three-dimensional model of the surface of the retina, or other three-dimensional representation of the retina 300.

One or both of the pre-operative images from 402 and the pre-operative three-dimensional geometry of the retina 300 may include reference markers, labels, and/or other information that may be used in subsequent registration. For example, a center of the fovea, lines or other features for establishing an orientation, or other markings may be included.

The method 400a may include projecting, at 406, a mesh, such as the mesh 312, onto the retina 300. The mesh may be projected using the endoillumination probe 208, a projector incorporated into the ophthalmic microscope 102, or some other light source. 406 may further include registering the mesh 312 with respect to the three-dimensional geometry of the retina 300. For example, 406 may include capturing images of the retina 300 using one or both of the cameras 204a, 204b; obtaining a three-dimensional image of the retina 300 from the images; registering the three-dimensional image with respect to the pre-operative three-dimensional geometry from 404; and projecting the mesh 312 onto the retina 300 with alignment according to the registering. For example, the mesh 312 may be aligned with or centered using reference markers, or other labels associated with the pre-operative three-dimensional geometry. The location of the reference markers or other labels relative to the retina 300 may be determined using registration. For example, a transformation of the pre-operative three-dimensional geometry to align with the three-dimensional image (e.g., align veins, fundus, and other features) may be used to transform the reference markers or other labels into correct positions relative to the retina 300.

The method 400a may include capturing, at 406, images of the retina 300 with the mesh 312 being projected onto it. 406 may include capturing pairs of images using the left camera 204a and right camera 204b at each timestep of a plurality of timesteps. 406 may include capturing pairs of images repeatedly over time, e.g., a pair of streams of video images from the left camera 204a and right camera 204b. The pairs of images may be captured periodically, e.g., at the frame rate of the streams of video images. The pairs of images may each be processed as the current pair of images according to the method 400a.

The method 400a may include processing the current pair of images from 408 to detect, at 410, representations of the mesh 312 in the current pair of images and detect, at 412, deformation of the mesh relative to a current representation of the mesh 312 in the current pair of images.

For example, 410 may include, for the current pair of images, identifying the representations of the mesh 312 in each image of the current pair of images and processing the representations of the mesh 312 to obtain a current three-dimensional representation of the mesh 312. The current three-dimensional representation may be compared to a reference three-dimensional representation to determine deformation at 412. The reference three-dimensional representation may be either (a) obtained from a prior pair of images received from the left camera 204a and right camera 204b prior to any actions that could cause deformation of the retina 300 (e.g., prior to insertion of an instrument 304, prior to commencement of peeling, etc.), (b) obtained from the pre-operative three-dimensional geometry, or (c) obtained from a prior pair of images received from the left camera 204a and right camera 204b, such as the pair of images received immediately before the current pair of images. In the case of (b), the reference three-dimensional representation of the mesh 312 may be virtually generated, e.g., a simulated projection of the mesh onto the three-dimensional geometry.

Determining the deformation at 412 may include determining local variation in three-dimensions between the current three-dimensional representation and the reference three-dimensional representation. For example, the three-dimensional positions of each vertex (e.g., intersection of grid lines) of the mesh 312 in the current and reference three-dimensional representations may be identified and the difference in the positions of the vertex may be used as the deformation for that vertex.

The deformation as determined at 412 may be processed according to the method 400a. In some embodiments, deformation determined using the approach described above with respect to FIGS. 3A to 3D may also be processed according to the method 400a.

The method 400a may include deriving, at 414, stress on the retina 300 from the deformation determined at 412. For example, the amount of stress experienced by the retina 300 may be estimated based on the deformation (i.e., strain) measured from 412 and estimates (e.g., experimentally determined estimates) for the modulus of elasticity of the retina 300. 414 may include performing finite element method (FEM) using the measured deformation and experimentally determined physical properties of retinal tissue, particularly the modulus of elasticity. The FEM algorithm used may be an explicit algorithm that is guaranteed to converge.

414 may include processing deformation from 412 for pairs of images for different timesteps. In particular, the velocity and acceleration of points on the retina 300 may be obtained by comparing the deformation evident in pairs of images for different timesteps. For example, for a plurality of points on the retina, e.g., at the intersection of the illustrated grid lines of the mesh 312, one or more vectors may be obtained, such as a displacement vector. Displacement vectors at a point of the plurality of points for adjacent timesteps may be processed to obtain a velocity vector for that point. Velocity vectors at a point of the plurality of points for adjacent timesteps may be processed to obtain an acceleration vector for that point. Some or all of these vectors may then be processed using an analytic technique such as FEM to derive stress on the retina, e.g., a stress vector at some or all of the plurality of points on the retina 300.

In some embodiments, the stress vectors, or data derived therefrom, is output to the surgeon. For example, a false color image or contour plot showing stress on the retina (e.g., the magnitude of the stress vectors), an alert indicating that stress at one or more points on the retina has exceeded a threshold, an output indicating that stress on the retina is still below the threshold, or other output may be provided to the surgeon.

In some embodiments, the stress vectors may be further processed, at 416, to calculate a vector along which force should be applied to the retina, such as a pulling force or a pushing force. For example, a pulling vector (e.g., a pulling vector 314 as shown in FIG. 3D) may define a direction to pull on a membrane such that stress on the retina is reduced. The pulling vector may, for example, be obtained by determining an adjustment to the direction and/or magnitude of a stress vector at a point such that the pulling vector will result in reduced stress, or stress in a more bearable direction, than the stress vector. The adjustment may be determined by performing FEM for the tissue surrounding the point to model the response of the tissue to various magnitudes and directions of force, and thereby determine a pulling vector that will reduce stress in the tissue surrounding the point to below a threshold stress.

A pulling vector is just one example of guidance that may be calculated. Other procedures may have corresponding guidance determined as part of the method 400a. For example, for a vitrectomy, a location may be determined at which the vitreous should be suctioned next to reduce stress on the retina. For example, using the stress vectors of 414, a relatively unstressed location on the retina may be identified as a suitable site to perform suctioning.

The method 400a may include outputting, at 418, guidance to the surgeon. The guidance may be output to a display device 118, display device 120, or a display device internal to the ophthalmic microscope 102. The guidance may include representations of the stress derived at 414, a representation of a pulling vector 314 along which force should be applied from 416, or other guidance determined at 416. In certain embodiments, one or more of these representations may be augmented over the real-time image of the retina that is being displayed on display device 118, display device 120, or a display device internal to the ophthalmic microscope 102. For example, the real-time image may be one of the current pair of images or a rendering of the current three-dimensional representation.

408-418 may be repeated any number of times throughout a procedure. For example, each pair of images in video streams from the left camera 204a and the right camera 204b being processed as the current pair of images, or every Nth pair of images, where N is an integer greater than one, may be processed according to 408-418. The current three-dimensional representation of the mesh derived from the current pair of images may be used as the reference three-dimensional representation in a subsequent iteration of 408-418. Note that it is impossible for a human mind to be able to perform 408-418 as described herein.

First, the nature of the data involved in 408-418 makes it unfeasible for the human mind to be able to perform such operations. For example, receiving or determining pre-operative three-dimensional geometry that may include a volumetric image of the retina, a three-dimensional model of the surface of the retina, or other three-dimensional representation of the retina, cannot practically be performed in the human mind. In another example, registering a mesh with respect to the three-dimensional geometry of the retina, as defined in 406, also cannot be performed in the mind. Detecting deformation of the mesh by comparing a current three-dimensional geometry to a reference three-dimensional representation similarly cannot be performed in the mind. Deriving stress, which may include performing FEM, as well as calculating pulling vectors by determining an adjustment to the direction and/or magnitude of a stress vector, are also inherently complex calculations involving complex three-dimensional data that cannot be performed in the mind. Further, presenting representations of the stress derived at 414, a vector along which force should be applied from 416, etc., which may involve augmenting such representations on an image of the retina are also inherently operations that cannot be mental processes.

Second, 408-418 are continuously repeated throughout a surgical procedure in real-time. As a result, performing these complex operations in real-time, involving complex data and images, such as three-dimensional models that cannot be processed in the mind, are outside the scope of mental processes.

Referring to FIG. 4B, in some embodiments, a method 400b may be performed without projecting a mesh 312 onto the retina 300 (the method 400a) or a virtual mesh (FIGS. 3A to 3D). The method 400b may include performing some or all of 402-408 as described above with respect to the method 400a. For example, the method 400b may include capturing, at 408, one or more images of the retina as described above, e.g., capturing a pair of images used as the current pair of images as discussed below.

The method 400b may further include generating, at 420, a three-dimensional model of the retina 300. For example, the current pair of images may be processed to generate a current three-dimensional representation of the retina 300, such as a volumetric image of the retina or a three-dimensional surface representing the retina 300.

The method 400b may include comparing, at 422, the current three-dimensional representation to a reference three-dimensional representation. The reference three-dimensional representation may include a prior three-dimensional representation from a previous iteration of 420, e.g., the current three-dimensional representation may be used as the reference three-dimensional representation in subsequent iterations of the method 400b. In such embodiments, in a first iteration of the method 400b, only 408 and 420 are performed since a reference three-dimensional representation does not yet exist.

422 may include registration whereby movement of the eye 106 relative to the left camera 204a and the right camera 204b is compensated for by transforming the reference three-dimensional representation to conform to the current orientation of the eye 106. 422 may include determining local deviations of the current three-dimensional representation relative to the reference three-dimensional representation. These local deviations may be interpreted as deformation of the retina 300 relative to the reference model.

For example, 422 may include dividing one or both of the current and reference three-dimensional representations into cells, e.g., cells including different areas of the surface of the retina as shown in the mesh of FIG. 3D. The cells may then be compared to one another to determine deformation evident in differences in the cells. For example, for each mesh position in the mesh, a cell at that mesh position in the current three-dimensional representation may be compared to the cell at that mesh position in the reference three-dimensional representation. Distortion and displacement of visible features of the cells relative to one another may be used to determine the change in position and shape (e.g., stretching) of the portion of the retina corresponding to that mesh position.

The deformation as determined at 422 may then be evaluated at 414 to determine stress on the retina 300. The process by which deformation is converted to stress at 414 may be as described above with respect to 414 of the method 400a. 416 and 418 may also be performed as part of the method 400b as described above.

408, 420, 422, and 414-418 may be repeated any number of times throughout a procedure. For example, each pair of images in video streams from the left camera 204a and the right camera 204b, or every Nth image, where N is an integer greater than one, may be processed as the current pair of images according to 408, 420, 422, and 414-418 of the method 400b. The current three-dimensional representation derived from the current pair of images, may be used as the reference three-dimensional representation in a subsequent iteration of 408, 420, 422, and 414-418.

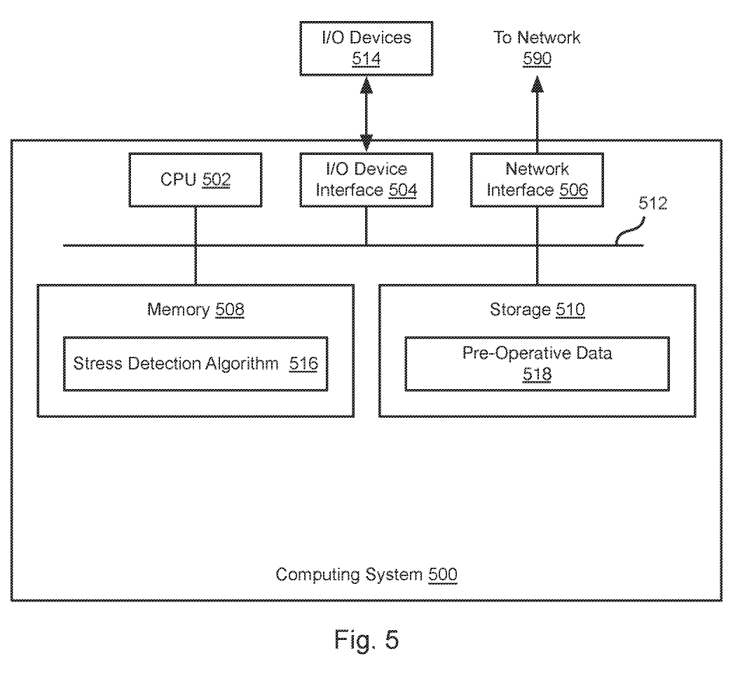

FIG. 5 illustrates an example computing system 500. The ophthalmic microscope 102 and/or the display device 120 may incorporate a computing device having some or all of the attributes of the computing system 500.

As shown, computing system 500 includes a central processing unit (CPU) 502, one or more I/O device interfaces 504, which may allow for the connection of various I/O devices 514 (e.g., keyboards, displays, mouse devices, pen input, etc.) to computing system 500, network interface 506 through which computing system 500 is connected to network 590, a memory 508, storage 510, and an interconnect 512.

CPU 502 may retrieve and execute programming instructions stored in the memory 508. Similarly, CPU 502 may retrieve and store application data residing in the memory 508. The interconnect 512 transmits programming instructions and application data, among CPU 502, I/O device interface 504, network interface 506, memory 508, and storage 510. CPU 502 is included to be representative of a single CPU, multiple CPUs, a single CPU having multiple processing cores, and the like.

Memory 508 is representative of a volatile memory, such as a random access memory, and/or a nonvolatile memory, such as nonvolatile random access memory, phase change random access memory, or the like. As shown, memory 508 may store executable code implementing a stress detection algorithm 516, such as an algorithm implementing one or both of the methods 400a and 400b.

Storage 510 may be non-volatile memory, such as a disk drive, solid state drive, or a collection of storage devices distributed across multiple storage systems. Storage 510 may optionally store pre-operative data 518, such as one more pre-operative images of the retina 300 and optionally three-dimensional geometry of the retina 300 as described above with respect to 404.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more operations or actions for achieving the methods. The operations and/or actions may be interchanged with one another without departing from the scope of the claims. In other words, unless a specific order of operations or actions is specified, the order and/or use of specific operations and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An ophthalmic surgical system comprising:
an ophthalmic microscope including a first camera and a second camera; and
a controller coupled to the first camera and the second camera, the controller configured to:
receive a current pair of ophthalmic images from the first camera and the second camera, the current pair of ophthalmic images including a current representation of a retina of a patient;
obtain a current three-dimensional representation of the retina from the current pair of ophthalmic images;
determine deformation of the retina by evaluating the current three-dimensional representation relative to a reference representation of the retina;
determine stress on the retina according to the deformation; and
output guidance according to the stress.

2. The ophthalmic surgical system of claim 1, wherein:
the reference representation includes a prior three-dimensional representation of the retina obtained from a prior pair of images captured using the first camera and the second camera prior to capturing the current pair of ophthalmic images; and
the controller is further configured to determine the deformation of the retina according to a comparison between the current three-dimensional representation of the retina and the prior three-dimensional representation.

3. The ophthalmic surgical system of claim 2, wherein the controller is further configured to:
identify first representations of a plurality of features in the prior three-dimensional representation;
identify second representations of the plurality of features in the current three-dimensional representation;
determine differences between the first representations and the second representations; and
determine the stress on the retina according to the differences.

4. The ophthalmic surgical system of claim 1, wherein the controller is further configured to:
for each region of a plurality of regions of the reference representation defined by a mesh:
identify one or more first representations of one or more features in the each region;
identify one or more second representations of the one or more features in the current three-dimensional representation;
determine one or more differences between one or more first representations and the one or more second representations;
determine strain for the each region according to the one or more differences; and
determine stress in the retina for the each region according to the strain.

5. The ophthalmic surgical system of claim 4, wherein the controller is further configured to determine stress in the retina for the each region according to the strain using finite element modeling (FEM).

6. The ophthalmic surgical system of claim 1, further comprising a light source configured to project a mesh onto the retina of the patient.

7. The ophthalmic surgical system of claim 6, wherein the controller is further configured to:
detect a current representation of the mesh in the current pair of ophthalmic images; and
determine the deformation of the retina according to the current representation of the mesh.

8. The ophthalmic surgical system of claim 1, wherein the controller is configured to determine stress on the retina according to at least one of velocity and acceleration of the deformation of the retina.

9. The ophthalmic surgical system of claim 1, wherein the guidance is a graphical representation of the stress.

10. The ophthalmic surgical system of claim 1, wherein the guidance is a vector along which to apply force to the retina.

11. A ophthalmic surgical method comprising:
receiving, by a controller, a current pair of ophthalmic images from a first camera and a second camera of an ophthalmic microscope, the current pair of ophthalmic images including a current representation of a retina of a patient;
obtaining, by the controller, a current three-dimensional representation of the retina from the current pair of ophthalmic images;
determining, by the controller, deformation of the retina by evaluating the current three-dimensional representation relative to a reference representation of the retina;
determining, by the controller, stress on the retina according to the deformation; and
outputting, by the controller, guidance according to the stress.

12. The ophthalmic surgical method of claim 11, further comprising projecting, by a light source of the ophthalmic microscope, a mesh onto the retina of the patient.

13. The ophthalmic surgical method of claim 12, further comprising:
detecting, by the controller, a current representation of the mesh in the current pair of ophthalmic images; and
determining, by the controller, the deformation of the retina according to the current representation of the mesh.

14. The ophthalmic surgical method of claim 13, wherein:
the reference representation includes a prior representation of the mesh in a prior pair of images captured using the first camera and the second camera prior to capturing the current pair of ophthalmic images; and
the ophthalmic surgical method further comprises determining, by the controller, the deformation of the retina according to the current representation of the mesh by comparing the current representation of the mesh with a prior representation of the mesh.

15. The ophthalmic surgical method of claim 13, wherein the reference representation includes three-dimensional geometry of the retina in pre-operative data.
